(19) United States
(12) Patent Application Publication
(10) Pub. No.: US 2013/0065260 A1
(43) Pub. Date: Mar. 14, 2013

(54) Compositions, Methods and Uses for Simultaneous Assay of Thrombin and Plasmin Generation

(71) Applicants: Mindy Grunzke, Denver, CO (US); Neil Goldenberg, Aurora, CO (US); Marilyn J. Manco-Johnson, Greenwood Village, CO (US); Linda Jacobson, Denver, CO (US); William E. Hathaway, Denver, CO (US)

(72) Inventors: Mindy Grunzke, Denver, CO (US); Neil Goldenberg, Aurora, CO (US); Marilyn J. Manco-Johnson, Greenwood Village, CO (US); Linda Jacobson, Denver, CO (US); William E. Hathaway, Denver, CO (US)

(73) Assignee: The Regents of the University of Colorado, a body corporate, Denver, CO (US)

(21) Appl. No.: 13/508,341

(22) PCT Filed: Nov. 5, 2010
(86) PCT No.: PCT/US10/55726

§ 371 Date: Nov. 20, 2012

Related U.S. Application Data
(60) Provisional application No. 61/259,034, filed on Nov. 6, 2009.

(51) Int. Cl. C12Q 1/56; C12M 1/34; G01N 21/64

(52) U.S. Cl. 435/13; 435/287.1

(57) ABSTRACT

Embodiments of the present invention report compositions and methods of analyzing thrombin and plasmin generation in a sample from a subject. In certain embodiments, the methods may comprise introducing at least one activator of coagulation and at least one activator of fibrinolysis to a sample and analyzing the sample for kinetic parameters related to thrombin and plasmin generation. In other embodiments, assays disclosed herein concern assessing net hemostatic balance of a subject for diagnostic and/or therapeutic applications.

### RELATED APPLICATIONS

The present application claims the benefit under 35 U.S.C. §119(e) of provisional U.S. patent application entitled “Compositions, Methods and Uses for Simultaneous Assays of Thrombin and Plasmin,” Ser. No. 61/259,034, filed on Nov. 6, 2009. This application is incorporated herein by reference in its entirety for all purposes.

### FIELD

Embodiments of the present invention generally relate to compositions, methods and uses for combined assessment of thrombin generation and plasmin generation in a sample from a subject. Some embodiments concern simultaneously assessing thrombin generation and plasmin generation in a sample from a subject. Methods herein can be used to assess a subject's prothrombotic and/or hemorrhagic tendencies that may be of significance in a wide variety of medical situations. Other embodiments report assays disclosed that concern assessing net hemostatic balance of a subject for diagnostic and/or therapeutic applications.

### BACKGROUND

Predicting and preventing catastrophic bleeding or excessive clotting (“thrombotic”) episodes in patients with coagulation disorders remains a critical, and largely unrealized, medical challenge. Despite many scientific advances in recent years to better understand bleeding and thrombotic disorders on the level of gene mutations, such diseases continue to cause long-term disability in a significant subset of patients. Ability to predict catastrophic bleeding or clotting episodes is an important goal for patients and their treating health professionals in order to maximize the potential for an enduring high level of patient functioning.

The present emphasis on further elucidating the molecular basis of coagulation diseases, while essential to the development of more targeted therapeutic approaches, has to date inadequately addressed many important questions that continue to complicate patient care on a daily basis.

Since the understanding of bleeding and thrombotic disorders has had an increasing molecular emphasis, the number of identifiable disorders of hemostasis has expanded. But, patients with similar molecular defects often have considerable variation in clinical phenotype. Therefore, these patients need better management guidelines and recommendations once diagnosed related to management, prophylaxis and treatment to improve their care.

### SUMMARY

Embodiments of the present invention report methods, compositions and uses for evaluating generation of thrombin and plasmin in one or more samples from a subject having or suspected of developing a blood disorder. In certain embodiments, compositions, methods and uses concern simultaneous evaluation of generation of thrombin and plasmin capacity found in one or more samples from a subject. In one exemplary assay, simultaneous thrombin and plasmin generation capacities are assessed over the course of clot formation and clot lysis of a sample. In accordance with these embodiments, coagulation is activated and fibrinolysis is accelerated in vitro. In one example assay, independent detectible or traceable agents can be used to detect thrombin and plasmin. In accordance with these embodiments, a detectible agent can be a fluorometric substrate.

In certain embodiments, raw measurements of, and kinetic parameters for, plasmin and thrombin generation can be used to assess a subject's net hemostatic balance at a given time, allowing prothrombotic and hemorrhagic risk assessment. In some embodiments, raw measurements can include maximum amplitude (MA) of fluorometric intensity, time to onset of rise in intensity beyond a given threshold (T_{lag}), time to maximum intensity (Tₘₐₓ), maximum slope of rise in intensity (v₁ₘₐₓ), average slope of rise in intensity (v_{1avg}), time to maximum slope of rise in intensity (Tv₁ₘₐₓ), and the area under the curve (AUC) over measured time intervals. In accordance with these embodiments, thrombin and plasmin generation capacities can be evaluated as they relate to normal and abnormal states of coagulation and fibrinolysis in adolescent and mature subjects.

In certain embodiments, involving simultaneous measurement of thrombin and plasmin generation in a sample, information obtained can be more comprehensive and more directly related to actual physiological conditions of a subject for clot formation and clot lysis in the body than most available assays. The disclosed methods and compositions allow rapid and inexpensive assessment of the hemostatic balance in an individual over time.

In certain embodiments, simultaneous assessment of thrombin and plasmin generation capacities of one or more samples from a subject can be used to diagnose a blood disorder in the subject or assess treatment for a subject having a blood disorder. Because subjects having the same blood disorder can have considerable variability among the subjects, understanding overall clot formation and lysis capabilities in a subject can lead to more tailored monitoring and treatment of the subject. Some embodiments concern collecting samples from a subject suspected of having a blood disorder and comparing them to a normal subjects sample (not having a blood disorder) for diagnosis. Other embodiments concern collecting samples from a subject and comparing them to other subjects affected by the same disorder in order to assess progression of the disorder and/or evaluate a treatment regimen.

Blood disorders completed herein include, but are not limited to, factor V Leiden mutation, prothrombin 20210 mutation, native anticoagulant deficiency, protein C deficiency, protein S deficiency, protein Z deficiency, protein Z-dependent inhibitor deficiency, antithrombin deficiency, activated protein C resistance, factor IIa excess, factor VII excess, factor VIII excess, factor IX excess, factor XI excess, other coagulation factor excess, lupus anticoagulant, anticardiolipin antibodies, beta-2 glycoprotein-1 antibodies, other antiphospholipid antibodies, elevated plasma or serum levels of homocysteine, elevated plasma or serum levels of lipoproteins, elevated plasma or serum levels of lipoprotein a, dyslipidemia, hypercholesterolemia or other similar blood disorder known in the art. Other embodiments can include, but are not limited to, blood disorder deficiencies, prothrombin deficiency, factor VII, factor VIII, factor IX, factor X, plasminogen, fibrinogen deficiencies or other deficiencies. In certain embodiments, blood disorder deficiencies can have varied levels of plasmin and thrombin capacities that can be reflective of a particular disorder.

Measurement of thrombin and plasmin generation may be performed in a container or test cell, including but not limited to 96-well microtiter plates, into which the sample (e.g. fresh or freeze-thawed, platelet-poor or platelet-rich plasma) and appropriate reagents have been added. An exemplary apparatus of use may include a sample, one or more reagents, buffer, fluorescent substrate, a reagent chamber, and a detection instrument such as a fluorescent spectrophotometer. In some embodiments, reagents added to a chamber may include small amounts of tissue factor (TF) and/or tissue-type plasminogen activator (tPA). In containers with multiple compartments, a sample may be analyzed in replicates, such as duplicate wells of a 96-well plate. In certain embodiments, an advantage of the disclosed methods is that the amount of sample required to assay can be relatively small, such as 90 μL of plasma per well.

DESCRIPTION OF ILLUSTRATIVE EMBODIMENTS

### BRIEF DESCRIPTION OF THE FIGURES

The following drawings form part of the present specification and are included to further demonstrate certain embodiments of the present invention. The embodiments may be better understood by reference to one or more of these drawings in combination with the detailed description of specific embodiments presented herein.

FIGS. 1A-1D represents normal (control) adult thrombin and plasmin generation curves with defined parameters, (A) Thrombin generation; (B) Velocity of thrombin generation; (C). Plasmin generation; and (D). Velocity of plasmin generation.

FIG. 2 represents an example of thrombin curves of plasma from a pregnant woman heterozygous for factor V Leiden (hypercoagulable state) and a man with severe factor VIII deficiency (hypocoagulable state), both in comparison to pooled normal plasma of healthy non-pregnant adults.

FIG. 3 represents an example of velocity of thrombin generation as curves from a pregnant woman heterozygous for factor V Leiden (hypercoagulable state) and a man with severe factor VIII deficiency (hypocoagulable state), both in comparison to pooled normal plasma of healthy non-pregnant adults.

FIG. 4 represents STP curves of initiation of coagulation defects Factor VII deficiency (<1%) and Factor XII deficiency (<1%) compared to a control.

FIG. 5 represents some defects of propagation of coagulation related to—Factor X deficiency (<1%), Factor VIII deficiency (<1%), and Prothrombin deficiency (<1%).

FIG. 6 represents an example of plasmin generation curves of plasma from alpha-2-antiplasmin deficient state (hyperfibrinolytic state) and plasminogen deficient state (hypofibrinolytic state), both in comparison to pooled normal plasma of healthy adults.

FIG. 7 represents exemplary data collected of thrombin and plasmin generation related to defects of fibrinolysis including deficiencies of fibrinogen (<15 mg/dL), plasminogen (<1%), prothrombin (<1%), and Alpha-2-antiplasmin (A2AP) (<1%).

FIGS. 8A-8D represent STP curves of mixing studies; (A). Factor VIII deficiency (50%, 12.5%, 3%, <1%). (B). Prothrombin deficiency (50%, 30%, 10%, <1%). (C). Fibrinogen deficiency (347 mg/dL, 120 mg/dL, 26 mg/dL, 15 mg/dL), and (D). Plasminogen deficiency (50%, 30%, 10%, <1%).

FIGS. 9A-9C represent influence of (A) factor VIII activity on the velocity of thrombin generation as a curve, (B) fibrinogen concentration on the velocity of plasmin generation as a curve, and (C) plasminogen concentration on the velocity of plasmin generation as a curve.

### DEFINITIONS

Terms that are not otherwise defined herein are used in accordance with their plain and ordinary meaning

As used herein, “a” or “an” can mean one or more than one of an item.

As used herein, “modulation” refers to a change in the level or magnitude of an activity or process. The change may be either an increase or a decrease. For example, modulation may refer to either an increase or a decrease in activity or levels. Modulation may be assayed by determining any parameter that indirectly or directly affects or reflects a condition.

As used herein, “capacity” can be overall capacity and can be monitored over a predetermined period of time. Capacity can be determined by rate, amplitude and amounts or other parameters disclosed herein, for example, for plasmin and/or thrombin.

### DETAILED DESCRIPTION

In the following sections, various exemplary compositions, methods and uses are described in order to detail various embodiments. It will be obvious to one skilled in the art that practicing the various embodiments does not require the employment of all or even some of the details outlined herein, but rather that concentrations, times, temperature and other details may be modified through routine experimentation. In some cases, well known methods or components have not been included in the description.

In accordance with embodiments of the present invention, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

With emphasis on further elucidating molecular basis of coagulation diseases, while essential to the development of more targeted therapeutic approaches, this has inadequately addressed many important questions that continue to complicate patient care on a daily basis. Clinicians are still unable, for example, to distinguish among hemophilia B patients with similar factor IX levels those patients who are at greatest risk for clinically-significant bleeding and who may therefore benefit from aggressive prophylactic or therapeutic interventions. Despite much progress on the molecular level in the field of thrombophilia research, most recently with the identification of the Factor V Leiden and prothrombin 20210 mutations, many patients with thrombosis have no detectable thrombophilia trait. Even more numerous are patients who have one or more identifiable thrombophilia traits. For these patients, there is as little medical understanding of composite prothrombotic risk upon which to guide management decisions regarding thromboprophylaxis and antithrombotic therapy. Embodiments disclosed herein concern assessing plasmin and thrombin capacities in a subject having or suspected of developing a blood disorder in order to further define clinically relevant physiological parameters of the subject for diagnosis and treatment.

Thrombin and plasmin are the key enzymes involved in coagulation and fibrinolysis, respectively. Plasma coagulative and fibrinolytic potentials in normal children and adults, and in representative pathologically altered hemostatic states, have been evaluated via simultaneous assessment of thrombin and plasmin generation. In some embodiments, such a method offers unique potential for understanding relative coagulative and fibrinolytic capacities in physiologically and pathologically altered hemostasis of a subject.

Unlike panels of individual molecular tests, assays that evaluate net clotting potential, or the generation of key enzymes in the coagulation system (e.g., thrombin and plasmin), can provide a more complete fingerprint of a subject's clotting state. Global tests have been attempted, but their clinical utility has most often been impeded by concerns of physiologic relevance, reproducibility, complexity, cost, timely results, and the requirement for continuous or multiple blood sampling.

Among the classical global assays, only the thromboelastogram (TEG) and euglobulin lysis time (ELT) assay continue to be used clinically. A recent rise of interest in global tests of coagulation and fibrinolysis has brought attention to the need for global assays sensitive to an array of hemostatic alterations.

Existing thrombin generation assays, while providing an important representation of coagulability, do not assess the fibrinolytic activities, a component of hemostasis with important clinical relevance. Altered fibrinolysis has been demonstrated not only in the physiologic states of pregnancy and the neonatal period, but also has been implicated in numerous bleeding and prothrombotic conditions. For example, excessive fibrinolysis is observed in severe hemophilia A and hepatic cirrhosis and deficient fibrinolysis has been demonstrated in the context of renal failure and elevated plasma lipoprotein(a) levels.

Despite some advances, a need still exists for a global assay that measures both plasma coagulation and fibrinolytic capacity, over a continuous window that is suitable for both pediatric (including neonatal) and adult clinical applications. Such an assay could include measurement of key enzymes that mediate the coagulation and fibrinolytic functions and would allow evaluation of subject's unique net hemostatic balance at any given time, and a possibility of contributing to prothrombotic and hemorrhagic risk assessment and treatment. Certain embodiments herein employ compositions and methods for measuring both thrombin and plasmin generation in one or more samples from a subject. Thrombin and plasmin are key enzymes of coagulation and fibrinolysis (respectively). Other embodiments of the present invention report simultaneous measurement of thrombin and plasmin generation in one or more samples from a subject.

Use of compositions and methods disclosed herein represent methods of suitable precision (intra- and inter-assay CVs) in the measurement of key parameters of thrombin and plasmin generation capacity, and analytical sensitivity to a broad array of single-factor alterations in hemostasis. Some of these alterations include, but are not limited to, an initiation phase of thrombin generation, a thrombin propagation phase, and a fibrinolytic system. Relationships between thrombin and plasmin generation capacities encompassed by compositions and methods disclosed herein can distinguish normal from various abnormal states in a subject or mildly abnormal to very abnormal states in a subject. In accordance with these methods, embodiments reported herein can be used to identify a delay in both thrombin and plasmin generation in severe factor VII, VIII, IX, and X deficiencies as compared to normal curves in healthy individuals and factor XII deficiency or differences among subjects having these deficiencies. Certain embodiments disclosed herein may be used to identify a subject having these disorders for diagnosis, analysis and/or treatment.

Other embodiments concern using compositions and methods disclosed herein to diagnose a child versus and adult. STP assay findings suggested that pediatric hemostatic balance differed from adults, characterized by a symmetric delay in onset, and decrease in rate and amount, of the generation of both thrombin and plasmin. Thus, compositions and methods disclosed herein as applied to a child could potentially differ from an adult. For example, proper controls involving normal healthy children and/or controls involving abnormal samples from a child can be used to assess whether a child suspected of having a blood disorder (e.g. Factor VII, VIII etc) has the disorder, assess progression of a disorder or responds to a predetermined treatment to attenuate a known blood disorder.

### General Considerations for Clotting and Fibrinolysis Assays

The coagulation and fibrinolysis systems are extraordinarily complex and interwoven processes that involve dozens of proteins, each of which may become dysfunctional due to genetic variation, traumatic injury and/or a disease state. Traditionally-used coagulation tests include those such as a PTT (activated partial thromboplastin time) which does not disclose events occurring at the molecular level. Both the positive and negative dynamics of clotting must be understood for optimal care of a subject in order to fully understand a subject's coagulation and fibrinolysis overall health and prescribe the correct treatment.

Certain embodiments herein report methods and compositions to simultaneously analyze the generation of thrombin and plasmin, the key catalysts of coagulation and fibrinolysis (respectively). An inexpensive and reliable assay evaluating both thrombin and plasmin generation will promote optimal application of physicians' resources to diagnose particular blood factor deficiencies and other conditions of altered hemostasis, monitor response to drug regimen and enhance treatment efficacy, leading to a decreased loss of life and decreased cost.

In certain embodiments, in order to accomplish in vitro measurement of thrombin and plasmin generation, traces of additional reagents may be added to the sample (e.g. blood sample) to induce or maximize clot formation and clot lysis in the mixture. These reagents may include predetermined amounts of TF and/or tPA (tissue-type plasminogen activator) or other known activators of clot formation and/or lysis.

Methods and compositions of assays of simultaneous thrombin and plasmin generation (STP) are disclosed herein developed to measure thrombin and plasmin in blood samples (e.g. plasma) using individual fluorometric substrates. Coagulation can be initiated with dilute tissue factor, phospholipid, and calcium in platelet-poor plasma; fibrinolysis can be accelerated via tissue plasminogen activator (tPA). Abnormal states of hemostasis were investigated. Some embodiments herein concern native plasma. Other embodiments concern using platelet poor plasma. Any method known in the art for collecting and preparing blood samples are considered of use in methods disclosed herein. In certain embodiments, collection and preparation of plasma samples from adults and children are contemplated for methods described herein.

In accordance with these methods and compositions, STP assays reproducibility and normal adult and pediatric values for measured and calculated parameters have been established. Onset of both thrombin and plasmin generation was significantly delayed in children relative to adults (p<0.001) and the maximum amplitudes of thrombin and plasmin generation were less in children than adults (p<0.01). It was observed that no significant differences were measured among pediatric age groups tested. Some impairments in thrombin generation were observed for extrinsic and common pathway factor deficiencies. In other embodiments, plasmin generation can be impaired in deficiencies of fibrinogen and plasminogen as well as with decreased tPA reagent concentration and addition of aminocaproic acid. In certain embodiments, plasmin generation can be enhanced by alpha-2-antiplasmin deficiency and excess tPA reagent. Simultaneous assessment of thrombin and plasmin generation in plasma can provide an enhanced understanding of overall coagulative and fibrinolytic functions in physiological and pathologically altered states of hemostasis in children and adults not previously observed using other assay systems known in the art.

### Advantages of the Assay

Some advantages of assays disclosed herein include, but are not limited to, reliable and reproducible results, and relatively inexpensive and readily available reagents. Additionally, certain equipment that can be used to monitor thrombin and plasmin generation, for example a fluorometric spectrophotometer, is already commercially available and does not require any extensive training of the operator. STP assays are very sensitive and requires a short time period to obtain results. Some embodiments report methods for producing results with assays disclosed herein around four hours or less. In certain embodiments, assays that measure thrombin and plasmin generation through clot formation and lysis provide more thorough individual information for a subject when compared to presently used thrombin generation or global clotting assay methods.

In certain embodiments, assays of disclosed herein continuously assess thrombin and plasmin generation. Fibrin is the key structural element of a venous clot, and is largely influence by the balance in amounts of thrombin (which generates fibrin) and plasmin (which breaks down fibrin). In accordance with these embodiments, certain assays disclosed herein independently measure parameters of coagulation activation and fibrinolysis. Other embodiments herein assess thrombin and plasmin generation by measuring a traceable agent associated with these molecules (e.g. fluorescence) in a sample. In various embodiments, assays herein can employ a first derivative of raw thrombin and plasmin curves, to obtain several parameters that can include, but are not limited to, peak velocity of thrombin generation; time to peak velocity of thrombin generation; peak velocity of plasmin generation and time to peak velocity of plasmin generation. In addition, these assays can analyze plasmin generation as a measure of fibrinolysis in disorders where fibrin clot formation is greatly impaired (e.g., severe deficiencies of factor II, V, VII, VIII, IX, IX or fibrinogen). This is in contradistinction to assays that measure fibrin formation, in which the development of a sufficient amount of fibrin clot is prerequisite to the measurement of clot breakdown. In other embodiments, timing of onset and maximal velocity of thrombin generation relative to timing of onset and maximal velocity of plasmin generation can be evaluated, as an index of relative balance of coagulation activation and fibrinolytic functions. For example, evaluation of these parameters can be performed at any or all time points selected.

### Disadvantages of Present Assay Systems

One present assay system, thromboelastography (TEG), uses whole blood and is available at point of care (POC) facilities. However, it focuses on the mechanical characteristics of a clot during its formation and dissolution, rather than measuring changes in the generation of key coagulative and fibrinolytic enzymes over time. In addition, the TEG technology is limited by typically requiring a fresh blood specimen. Furthermore, the TEG typically uses a non-physiological activation of coagulation (celite), rather than the physiological activator (tissue factor and phospholipid).

### Fibrinolytic Capacity Assessment

Some embodiments include compositions and methods for measuring both thrombin and plasmin in a subject having undergone a traumatic physiological event in order to evaluate a treatment regimen for the subject. Whether or not cell destruction can be minimized after traumatic physiologic events, for example, myocardial infarctions, stroke, or gangrene infection may depend, in part, upon the existence and understanding of pathologically or therapeutically induced fibrinolysis. In order to reduce or eliminate cell destruction in a subject who has undergone or is undergoing such a traumatic event, quickly ascertaining whether the subject's fibrinolytic capacity is within a normal range can be an important factor for prognosis and treatment of the subject. By comparing the subject's specific plasmin generation curve/parameters to either that of a control subject's physiologic states or within a given control subject over time, medical personnel may be able to determine whether the subject's specific fibrinolytic capability needs to be modified or otherwise taken into consideration.

Under conditions when arterial or venous thrombosis has occurred or is likely to occur, such as during and after surgery, medical personnel need reliable information available about a subject's fibrinolytic processes. For example, clot formation often occurs during cardiac surgery utilizing extra-corporeal passage of blood. Although clotting during cardiac surgery may be minimized through use of heparin or other anticoagulants, a surgical subject's natural lytic ability can help avoid surgical complications by dissolving any clots that form. If a particular surgical subject's lytic ability is impaired, medical personnel may elect to administer thrombolytic agents to maintain a particular level of lytic activity to reduce or avoid possibilities of permanent and disabling clot formation occurring during surgery. To maintain a desired level of lytic activity, it may be useful to detect whether the administration of a thrombolytic agent had desired effect upon the surgical subject.

In other aspects, when a deep venous thrombosis or pulmonary embolism is veno-occlusive and/or extensive in a subject, compromising venous or pulmonary function or risking chronic venous insufficiency due to venous valvular damage, thrombolytic therapy may be indicated. Such therapy would be better monitored (and its bleeding complications potentially minimized) through the use of an assay designed to measure fibrinolytic capacity of plasma at a given time, whether pre-treatment, during treatment, or post-treatment. Evaluation of plasmin generation can serve as a reliable and clinically useful surrogate for fibrinolytic capacity.

### Coagulation Potential Assessment

In other embodiments, subjects having or suspected of developing a bleeding disorder or other coagulation factor deficiencies may be evaluated by measuring the subject's coagulation potential for example, to tailor dose intensity and duration of therapies and/or prophylactic measures (e.g., the administration of factor concentrates or recombinant proteins) to the type and severity of the disturbance in coagulability exhibited by the subject's plasma at the time of the assessment and intervention. In yet other embodiments, prothrombotic conditions can be assessed in the subject by measuring the subject's coagulative capacity for example, to tailor dose intensity and duration of antithrombotic therapies and/or prophylactic measures (e.g., the administration of anticoagulants or thrombin inhibitors) to the type and severity of the subject's hypercoagulable state.

### Diseases and Conditions

Several bleeding disorders exist that can result from defects in the process of hemostasis. These bleeding conditions have been identified at the level of the proteins of the clotting cascades, platelet activation and function, contact activation and antithrombin function. Some of these disorders include, but are not limited to, Hemophilia A, or classic hemophilia (a disease referring to the inability to clot blood), is an X-linked disorder resulting from a deficiency in factor VIII, a key component of the coagulation cascade. There are severe, moderate and mild forms of hemophilia A that reflect the level of active factor VIII in the plasma. Hemophilia B results from deficiencies in factor IX. At least 300 unique factor IX mutations have been identified, 85% are point mutations, 3% are short nucleotide deletions or insertions and 12% are gross gene alterations. Clinical management of hemophilia B is complicated by the fact that the genotype and activity level of factor IX do not necessarily correlate with bleeding phenotype. Several cardiovascular risk factors are associated with abnormalities in fibrinogen. As a result of the acute-phase response or through other poorly understood mechanisms, elevated plasma fibrinogen levels have been observed in patients with coronary artery disease, diabetes, hypertension, peripheral arterial disease, thrombosis, hyperlipoproteinemia and hypertriglyceridemia. In addition, pregnancy, menopause, hypercholesterolemia, use of oral contraceptives and smoking lead to increased plasma fibrinogen levels.

Although rare, there are inherited disorders in fibrinogen. These disorders can include, but are not limited to, afibrinogenemia (a complete lack of fibrinogen), hypofibrinogenemia (reduced levels of fibrinogen) and dysfibrinogenemia (presence of dysfunctional fibrinogen). Afibrinogenemia is characterized by neonatal umbilical cord hemorrhage, ecchymoses, mucosal hemorrhage, internal hemorrhage, and recurrent abortion. The disorder is inherited in an autosomal recessive manner. Hypofibrinogenemia is characterized by fibrinogen levels below 100 mg/dL (normal is 250-350 mg/dL) and can be either acquired or inherited. Symptoms of hypofibrinogenemia are similar to, but less severe than, afibrinogenemia. Dysfibrinogenemias are extremely heterogeneous affecting any of the functional properties of fibrinogen. Clinical consequences of dysfibrinogenemias can include hemorrhage, spontaneous abortion, and thromboembolism. Early diagnosis of these conditions in a subject could lead to intervention and reduction or elimination of some of these conditions in the subject.

Factor XIII is the proenzyme form of plasma transglutaminase and is activated by thrombin in the presence of calcium ions. Activated factor XIII catalyzes the cross-linking of fibrin monomers. Factor XIII is a tetramer of two different peptides, a and b (forming a₂b₂). Hereditary deficiencies (autosomal recessive) result in the absence of either subunit. Clinical manifestation of factor XIII deficiency is delayed bleeding although primary hemostasis is normal. Deficiency leads to neonatal umbilical cord bleeding, intracranial hemorrhage, and soft tissue hematomas.

Von Willebrand disease (vWD) is due to an inherited deficiency in von Willebrand factor (vWF). vWD is the most common inherited bleeding disorder of humans. Using laboratory testing, abnormalities in vWF can be detected in approximately 8000 people per million. Clinically significant vWD occurs in approximately 125 people per million. This is a frequency at least twice that of hemophilia A.

Native (e.g. intrinsic) anticoagulants include antithrombin and proteins C and S. Clinical manifestations of native anticoagulant deficiency include deep vein thrombosis and pulmonary embolism. Thrombosis may occur spontaneously or in association with surgery, trauma, or pregnancy. Treatment of acute thrombotic episodes is most often by intravenous infusion of unfractionated heparin or subcutaneous administration of low-molecular-weight heparin (for 5-7 days) followed by oral anticoagulant therapy for at least 3-6 months. In the case of a persistent underlying risk factor, the treatment course may be longer (e.g., life-long in the setting of congenital anticoagulant deficiency). In addition, protein C and antithrombin concentrates may be used in subjects with inherited or acquired deficiencies of these proteins.

It would be useful for healthcare professionals to be able to quickly and accurately monitor a subject's total clot formation and lytic capacity, including both lysis resulting from natural fibrinolytic activity and from physiological responses to the therapeutic administration of thrombolytic agents. In addition, it would be useful to detect changes in lytic capacity caused by other therapeutically administered agents or by pathological conditions, including disseminated intravascular coagulation. In order to monitor blood condition changes caused by lytic activity, a test which evaluates plasmin generation in a sample of clotted blood in which lysis is allowed to proceed could be beneficial. However, one standard for fibrinolytic assessment, the euglobulin clot lysis assay (ECLA), also referred to as euglobulin lysis time (ELT), is limited to evaluating fibrin clot dissolution after key inhibitors of fibrinolysis have been removed from platelet-poor plasma.

Medical personnel have been hindered by an inability to prescribe individualized doses of thrombolytic or anti-fibrinolytic agents tailored to the unique physiological responses of a particular subject. Currently, no known tests are commercially available to determine the dose response to thrombolytic and anti-fibrinolytic agents. In the absence of such dose response data, a standardized dose is usually prescribed. A standardized dose may be either inadequate or excessive for a particular patient because of variations in body size, blood volume, blood chemistry, physiologic response, and pathological or surgical conditions. A rapid test to assess plasmin generation over time could be valuable for diagnosis and therapeutic monitoring.

### Thrombin and Plasmin Generation Assay

Some embodiments herein include obtaining one or more samples from a subject and simultaneously assaying thrombin and plasmin generation in the sample(s). In accordance with these embodiments, an assay may utilize buffered reactant solution containing for example, one or more trace amounts of one or more activators of coagulation, such as calcium, tissue factor (TF) and/or thrombin, and one or more activators of clot lysis, such as tissue-type plasminogen activator (tPA) (e.g. two-chain recombinant human tPA). TF (e.g. recombinant human TF) may be used in lipidated form for platelet-poor plasma assay and in non-lipidated form for platelet-rich plasma assay. Buffer solution can include, but is not limited to, Tris-buffered saline with calcium chloride. Other buffers known in the art can be used.

In certain embodiments, a buffered reactant solution may be added to a sample, such as fresh or freeze-thawed, platelet-poor or platelet-rich plasma in replicate wells of a 96-well assay plate. Samples may further comprise one or more cellular entities, such as white blood cells and/or endothelial cells, in suspension or in a monolayer. Samples can be analyzed by any detector known in the art capable of detecting traceable agents. In certain embodiments experimental reaction plates may be analyzed in an automated, thermo-regulated (37° C.), fluorescent spectrophotometer. The course of thrombin and plasmin generation may each be monitored as continuous changes in fluorescent intensities of a sample over time, for example, over about an hour, over about a couple of hours or over about four hours. In some embodiments, signal intensities of individual fluorometric substrates for thrombin and plasmin may be monitored continuously or at any pre-selected time frequency. Certain embodiments include having a spectrophotometer interface with a computer to permit analysis (e.g. real-time) of kinetic fluorometric intensity measurements using (a) data analysis program(s). In accordance with some of these embodiments, a thrombin generation curve can be analyzed along with a plasmin generation curve. A thrombin generation curve may be generated over the course of assay reactions that has an initial baseline intensity, followed by a progressive rise in signal to a point of maximum intensity, then completed by either a plateau or a progressive decline in intensity to a minimum or to baseline. Similarly, a plasmin generation curve may be generated over the course of the assay reactions that has an initial baseline intensity, followed by a progressive rise in signal to a point of maximum intensity, then completed by either a plateau or a progressive decline in intensity to a minimum or to baseline. A plasma standard (e.g. pooled plasma from healthy individuals) and controls (e.g. one normal and one to two abnormal controls) may be run simultaneously with the clinical/laboratory sample(s) using this protocol.

In some embodiments, specific measurements may include the maximum amplitude (MA) of fluorometric intensity, the time to onset of rise in intensity beyond a given threshold (T_{lag}), the time to maximum intensity (Tₘₐₓ), the maximum slope of rise in intensity (v₁ₘₐₓ), the average slope of rise in intensity (v_{1avg}), the time to maximum slope of rise in intensity (Tv₁ₘₐₓ), the area under the curve (AUC) over measured time intervals, and relative differences or ratios in these parameters when comparing thrombin and plasmin generation from the same specimen. Specimens may be compared between normal controls and patients suspected of having, or known to have, one or more pathologic conditions, such as hemophilia or other diseases relating to clot formation and/or clot lysis. In addition, assay results for controls and unknowns can be normalized to that of a simultaneously evaluated assay standard (e.g., pooled normal adult plasma).

Certain exemplary embodiments (see Examples) describe simultaneous thrombin and plasmin generation assays for assessing overall capacity in a subject. However, the skilled artisan will realize that concentrations of various reagents as well as times and temperatures of reactions may be varied from those specified below without undue experimentation. Furthermore, where various factors such as calcium, phospholipid, TF (e.g. lapidated TF), and tPA are disclosed, these may be substituted with alternative factors known to exhibit similar activities within the scope of claimed methods and compositions.

Assays disclosed herein are reproducible and analytically sensitive to deficiencies and excesses of key components in the coagulation and fibrinolytic systems, as well as to physiologic alterations in hemostasis. Measurement of these parameters may be applied to assess subjects with known and/or as yet undefined hemorrhagic and prothrombotic conditions.

In one embodiment, assay results may be analyzed in a subject suffering from a heart condition. Examples of heart conditions include, but are not limited to, myocardial ischemia, myocardial infarction, acute coronary syndromes, atherosclerotic coronary artery disease, valvular diseases, and congestive heart failure.

Other embodiments concern a subject having or suspected of developing a hepatic disorder.

In another embodiment, assay results may be analyzed in a subject having a prothrombotic condition. Examples of prothrombotic conditions include but are not limited to venous or arterial omboembolism (e.g. stroke), as well as hypercoagulable states (for example, factor V Leiden and prothrombin 20210 mutations, antiphospholipid antibodies, anticoagulant deficiency, and elevated levels of procoagulant factors, homocysteine, or lipoproteins).

In other embodiments, assay results may also be analyzed in subject having a bleeding condition. Examples of bleeding conditions include, but are not limited to, the hemophilias and other coagulation factor deficiencies or dysfunctions (including a/hypo/dysfibrinogenemia), von Willebrand disease, platelet function abnormalities, and fibrinolytic abnormalities (e.g. PAI-1 deficiency).

In yet another embodiment, assay results may be analyzed in healthy children and adults to assess bleeding and/or prothrombotic risk in the steady state and in times of altered (pathologic or physiologic) hemostasis, including the special physiologic states of pregnancy and the neonatal period. These assay results may be compared to healthy and abnormal samples in order to assess a subjects condition.

### Fluorometric Detection

Alternative fluorometric substrates and wavelength detections:

Certain embodiments herein can use fluorometric substrates capable of detection by a plate reader or other system capable of fluorescent detection and having variable wavelengths of which to detect a substrate used. In some embodiments fluorescence may be in the range of about 300 nm to about 500 nm for excitation and emission. It is contemplated herein that any fluorogenic substrate may be chosen for thrombin and plasmin generation detection. In addition, other methods and compositions for assessing plasmin and thrombin generation are contemplated, including, but not limited to, other molecules capable of emitting fluorescence such as tagged antibodies, antibody fragments, nucleic acids, aptamers or other small molecules capable of fluorescent excitation and emission or being tagged for such detection. Any traceable or detectible molecule capable of associating with plasmin or thrombin known in the art is contemplated.

Some embodiments concern measuring plasmin and thrombin generation in a sample at different times. Other embodiments concern compositions and methods for detecting only plasmin generation in one or more samples from a subject contemplated herein. In other embodiments, both thrombin and plasmin generation may be assessed in one or more samples. In accordance with these embodiments, thrombin and plasmin generation may be assessed simultaneously or thrombin generation may be assessed prior to plasmin. Other embodiments concern assessing initial concentrations of thrombin generation then assessing plasmin generation in one or more samples.

Data Generation and Detection and Analysis

Recording and plotting of captured information via computer interface:

In some embodiments, using a plate reader and its associated software, raw data reading (e.g. fluorescence) can be taken over predetermined intervals and/or periods. At the end of the reading time, the raw data can be exported into spreadsheet for further data analysis. In accordance with these embodiments, a spreadsheet can include formulas to automatically calculate specific parameters described herein. In addition to these parameters, these interfaces can plot graphs of the data for additional analysis and assessment. It is contemplated that predetermined ranges may be preprogrammed that alert a health professional when thrombin and/or plasmin generation are outside the predetermined range for a given subject. In accordance with these embodiments, a health professional my decide to treat, change a treatment or increase a treatment for a subject having such plasmin and/or thrombin concentrations.

### Medical Conditions

In certain embodiments, a subject that may benefit from compositions or methods disclosed herein may be a subject that has or is suspected of having an abnormal condition, comprising factor V Leiden mutation, prothrombin 20210 mutation, native anticoagulant deficiency, protein C deficiency, protein S deficiency, protein Z deficiency, protein Z-dependent inhibitor deficiency, antithrombin deficiency, activated protein C resistance, factor IIa excess, factor VII excess, factor VIII excess, factor IX excess, factor XI excess, other coagulation factor excess, lupus anticoagulant, anticardiolipin antibodies, beta-2 glycoprotein-1 antibodies, other antiphospholipid antibodies, elevated plasma or serum levels of homocysteine, elevated plasma or serum levels of lipoproteins, elevated plasma or serum levels of lipoprotein a, dyslipidemia, hypercholesterolemia, or a combination thereof. Other embodiments concern measuring thrombin and plasmin generation in certain cases for therapeutic monitoring of hemostatic (e.g., cryoprecipitate, fresh frozen plasma, factor concentrates), antithrombotic (e.g., heparin and its derivatives, warfarin, direct thrombin inhibitors, aspirin, clopidogrel), and thrombolytic (e.g., tissue plasminogen activator) therapies in humans or animals. Some embodiments concern other blood disorders such as factor deficiencies as disclosed previously.

Some embodiments concern methods for inhibiting contact activation of coagulation using corn trypsin inhibitor or other contact activation inhibitor. Other embodiments may further include adding predetermined amounts of heparin or heparin-like substances in order to enhance detection of antithrombin activity and its deficiency/excess. Yet other embodiments may further include adding an activator of the protein C pathway in order to enhance detection of abnormalities of the protein C pathway (e.g., protein C deficiency, protein S deficiency, factor V Leiden polymorphism).

Other embodiments concern methods and compositions of use in clinical research and studies involving a subject having or suspected of developing a blood disorder. In accordance with these embodiments, certain studies regarding thrombin and plasmin generation in these subjects can be compared within the different conditions and/or in relation to normal healthy controls.

### EXAMPLES

The following examples are included to demonstrate embodiments of the invention. It should be appreciated by those of skill in the art that the techniques disclosed in the examples which follow represent techniques discovered by the inventors to function in the practice of the invention, and thus can be considered to constitute modes for its practice. However, those of skill in the art should, in light of the present disclosure, appreciate that many changes can be made in the embodiments which are disclosed and still obtain a like or similar result without departing from the spirit and scope of the invention.

### Subject Groups

Plasma from healthy individuals without prior bleeding or thrombotic histories and not receiving anticoagulant, anti-platelet, or estrogen-containing medications was obtained commercially (Core Set Adult Normals, George King Bio-Medical, Inc., Overland Park, Kans.) for the establishment of physiologic assay values in adults. The median age of healthy adults (n=20 or n=30) was 35 years (range: 19-54 years).

All healthy individuals recruited for the establishment of normal values were screened by clinical history to exclude subjects with recent illness, prior abnormal bleeding or thrombotic events in the participant and first degree relatives, or recent use of antibiotic, anticoagulant, anti-platelet, or estrogen-containing medications. Samples from all healthy children and adults were confirmed to have prothrombin time (PT), activated partial thromboplastin time (aPTT), and fibrinogen values in the respective normal ranges. Plasmas from healthy adults were obtained commercially.

### Blood Collection and Sample Processing Procedures

Blood was collected with the child or adult participant at rest in the seated position by atraumatic peripheral venipuncture technique with minimal applied stasis into BD Vacutainer 3.2% buffered sodium citrate siliconized blood collection tubes (Becton-Dickinson, Franklin Lakes, N.J.), with collection of the initial 1 mL of blood into a discard tube. All specimens were centrifuged for 15 minutes at 4° C. and 2500×g, and the plasma supernatant was then centrifuged for an additional 15 minutes to remove any residual platelets. All samples were aliquoted into 1.5 mL copolymer polypropylene long-term freezer storage tubes with O-ring screw caps (USA Scientific, Ocala, Fla.) and stored at −70° C. until time of assay. Commercially-obtained individual and pooled platelet-poor plasma specimens (George King Bio-Medical, Inc., Overland Park, Kans.) were collected and processed by a similar protocol. Plasma found to be icteric or lipemic was excluded from analysis in assays that measure optical density or fluorescence intensity. Platelet-poor plasma (PPP) samples were aliquoted into tubes and stored at −70° C. until time of assay.

Simultaneous Thrombin and Plasmin generation (STP) Procedure

Frozen PPP aliquots are thawed in a 37° C. water bath for 3 minutes; comparison of freeze-thawed vs. fresh platelet-poor plasma specimens from the same person revealed no differences in the STP curves. Reactant solution is prepared as described previously for the CloFAL assay developed in our laboratory. Briefly, lipidated recombinant human tissue factor (TF) (American Diagnostica Inc, Stamford, Conn.), non-lipidated recombinant human TF (American Diagnostica Inc), and recombinant two-chain tissue plasminogen activator (tPA) (American Diagnostica Inc) are added to a stock solution of Tris-buffered saline (TBS; 66 mM Tris, 130 mM NaCl, pH=7.0) containing 34 mM CaCl₂, to a concentration of 10 pM of TF (made from 8.7 pM non-lipidated TF and 1.3 pM lipidated TF) and 900 ng/mL of tPA. Final concentrations are 5 pM TF and 450 ng/mL tPA after addition of reactant solution to plasma samples, as described below. The assay blank consists of TBS alone. The reactant solution is freshly prepared from stored stock solutions of TBS, aliquoted TF, and aliquoted tPA just prior to assay. Final concentrations of TF and tPA ranging from 0-10 pM and 0-900 ng/mL, respectively, were investigated during assay development, with ultimate concentrations of these reagents (as well as ratio of lipidated to non-lipidated TF) selected based upon empiric experiments designed to optimize precision and analytical sensitivity to pathologically and physiologically altered states of coagulation and fibrinolysis, as previously performed with the CloFAL assay

Two fluorometric substrates were used for the detection of the key enzymes, thrombin and plasmin. Boc-Val-Pro-Arg-MCA [t-Butyloxycarbonyl-L-Valyl-L-Prolyl-L-Arginine-4-Methyl-Coumaryl-7-Amide] is a fluorometric substrate for α-thrombin (Peptide Institute Inc, Osaka, Japan; 10 mM stock solution prepared according to manufacturer instruction) and Boc-Glu-Lys-Lys-MCA [t-Butyloxycarbonyl-L-Glutamyl-L-Lysyl-L-Lysine-4-Methyl-Coumaryl-7-Amide] is a fluorometric substrate for plasmin (Peptide Institute Inc, Osaka, Japan; 10 mM stock solution). Stock solutions of each fluorometric substrate are stored at −20° C. for up to one month. With each respective stock solution of fluorometric substrates, a fresh working solution of 1,000 μM concentration is made with dilution of the stock solution in TBS. Final well concentration of each fluorometric substrate is 100 μM

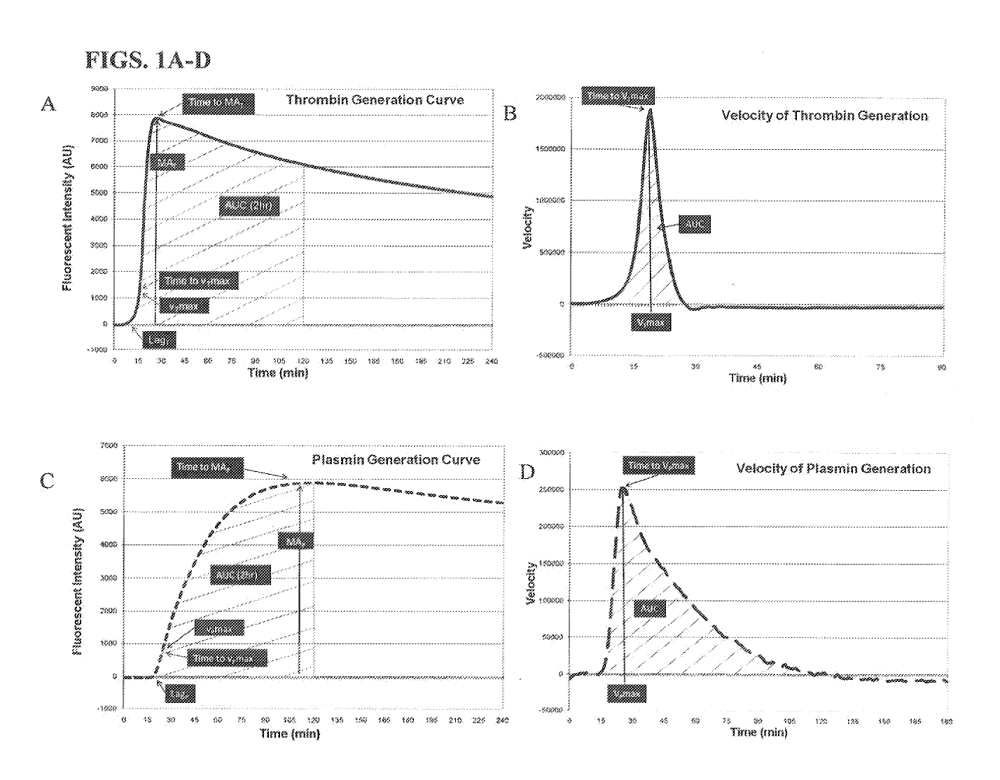

For each patient sample to be analyzed, 90 μL of reactant solution was added simultaneously to each well in a flat-bottom, black, 96 well Costar assay plate (Fisher Scientific, Santa Clara, Calif.) using a multi-tip automated pipette. Next, 20 μL of the appropriate fluorogenic substrate (thrombin or plasmin) was added to each well, where as an example the thrombin substrate is added to each well in row A of the 96-well plate while the plasmin substrate is added to each well in row B of the 96-well plate. This process was repeated for paired rows C-D, E-F, etc. The plate was then placed into the fluorescent spectrophotometer (Multi-Mode Microplate Reader Synergy 2, Bio-Tek Instruments, Winooski, Vt.) where it was pre-warmed at 37 ° C. for three minutes. At the end of the three minutes, 90 μL of freeze-thawed or fresh plasma sample was dispensed into each of four wells (two with the thrombin substrate, two with the plasmin substrate) using a multi-tip automated pipette. The plate was then drawn back into the fluorescent spectrophotometer where the sample is initially mixed for five seconds followed by fluorescent intensity measurements taken from each well every 45 seconds for four hours at a fluorescent emission of 360 nm and excitation of 460 nm (as determined by the chosen fluorescent substrates). The fluorescent spectrophotometer interfaced with a computer such that all its operations, including continuous analysis of fluorometric intensity data using GEN-5™ PC software, were automated. Represented in FIG. 1, a thrombin generation curve and separate plasmin generation curve were produced over the course of the assay reactions that had an initial baseline intensity, followed by a progressive rise to a point of maximum intensity, and completed by a progressive decline to minimum/baseline intensity. The kinetic fluorometric intensity data were exported to Microsoft Excel for additional data analyses in order to generate the aforementioned calculated parameters from the raw data. Mathematical formulas were programmed to allow automated report of measured and calculated assay parameters (e.g., results).

### Abnormal/Altered Plasma Experiments

Factor VIII deficient plasma was obtained from a patient with severe congenital deficiency, with a measured factor VIII activity of <1 U/dL. All other specific factor-deficient human plasmas were obtained commercially as snap-frozen specimens from patients with congenital factor deficiencies (Factor II, V, VII, IX, X, XI, XII, XIII, Prekallikrein, High-Molecular-Weight Kininogen (HMWK), and Fibrinogen Deficient Plasmas, George King Bio-Medical, Inc., Overland Park, Kans.). The activity level of the deficient factor was assayed at <1 U/dL in all cases, with the exception of factor II activity and fibrinogen concentration, which were 3 U/dL and <15 mg/dL, respectively. Additionally, immunodepleted plasmas were obtained to study aprothrombinemia (Haematologic Technologies Inc., Essex Junction, Vt.) and aplasminogenemia (Affinity Biologicals, Inc., Ancaster, ON, Canada). To test the analytic sensitivity of the assay to fibrinogen and factor VIII, fibrinogen-deficient plasma was mixed with standard normal pooled plasma to achieve final concentrations of 15, 26, 120, and 347 mg/dL, and factor VIII-deficient plasma was serially diluted with standard normal pooled plasma to achieve final concentrations of <1, 4, 14, 53, and 100 U/dL.

In altered fibrinolysis experiments, plasminogen-deficient plasma was mixed with standard normal pooled plasma to achieve final concentrations of 10%, 30%, and 50% of normal. To inhibit fibrinolysis, standard normal pooled plasma was treated with aminocaproic acid to achieve final plasma concentrations of 2.5 mg/mL and 0.25 mg/mL.

### Correlative Laboratory Assay Procedures

Prothrombin times (PT) were measured using Simplastin® Excel, and activated partial thromboplastin times (aPTT) using 0.025 molar calcium chloride and Automated APTT® reagent (bioMerieux, Inc., Durham, N.C.). Plasma fibrinogen concentration was determined by the clotting method of Clauss using Dade Behring thrombin and calibration reagents (Dade Behring, Marburg, Germany). Plasma factor VIII activity levels were ascertained with standard one-stage clotting assay with the same reagents as above for aPTT. All of these clotting assays were performed on the ST4 coagulometer (Diagnostica Stago, Asnieres-sur-Seine, France).

### Example 1

### Normal Thrombin and Plasmin Generation Curves in Adult Plasma

FIG. 1 represents exemplary plot of typical raw thrombin and plasmin generation curves for a healthy adult control. FIGS. 1A-1D represents normal (control) adult thrombin and plasmin generation curves with defined parameters, (A) Thrombin generation; (B) Velocity of thrombin generation; (C). Plasmin generation; and (D). Velocity of plasmin generation. The exemplary analytical technique used for these plots involves a standard normal pooled adult platelet-poor plasma specimen. The intra-assay coefficients of variation (CV) for the thrombin and plasmin generation assay were established for a normal control by using this standard along with 5 repeated samples of normal pooled plasma (Pooled Normal, George King Biomedical, Overland Park, Kans.). In each case, plasma samples were analyzed in duplicate on the same assay plate in a single run. Intra-assay CVs for normal controls were 1) thrombin parameters: T_{lag} 2.5%, MA 6.4%, Tₘₐₓ 2.1%, v₁ₘₐₓ 5.2%, Tv₁ₘₐₓ 3.3%, AUC over 2 hours 5.3% and 2) plasmin parameters: T_{lag} 0.7%, MA 4.8%, Tₘₐₓ 1.9%, v₁ₘₐₓ 4.7%, Tv₁ₘₐₓ 2.4%, AUC over 2 hours 4%. Inter-assay CVs, determined via serial testing of the normal standards on 20 separate runs, were 1) thrombin parameters: T_{lag} 21.3%, MA 4.7%, Tₘₐₓ 20.6%, v₁ₘₐₓ 17%, Tv₁ₘₐₓ 16.1%, AUC over 2 hours 4.8% and 2) plasmin parameters: T_{lag} 10.5%, MA 5.3%, Tₘₐₓ 14.5%, vᵢₘₐₓ 14.2%, Tvᵢₘₐₓ 11.6%, AUC over 2 hours 7.8%.

FIGS. 1A-1D represent observations recorded regarding normal adult thrombin and plasmin generation curves by STP assay. STP curves are labeled with measured and calculated parameters. (A). Thrombin generation; (B). Velocity of thrombin generation; (C). Plasmin generation; (D). Velocity of plasmin generation. Parameters include: Lag_{T}=thrombin lag time (time to start of thrombin generation), V_{T}max=maximum velocity of thrombin generation, time to V_{T}max=time to the maximum velocity of thrombin generation, MA_{T}=maximum amplitude of thrombin generation, time to MA_{T}=time to MA of thrombin generation, Lag_{P}=plasmin lag time (time to start of plasmin generation), V_{P}max=maximum velocity of plasmin generation, time to V_{P}max=time to the maximum velocity of plasmin generation, MA_{P}=maximum amplitude of plasmin generation, time to MA_{P}=time to MA of plasmin generation, AUC (2 hr)=area under the curve over the first 2 hours, AUC (velocity curves)=area under the velocity curve.

Unique thrombin and plasmin generation curves are produced (see for example FIG. 1). Each curve has an initial baseline fluorescent intensity, followed by a rise in fluorescent intensity (slope phase) to a maximum amplitude (MA), and completed by a plateau of the intensity (with some natural degradation of the maximum signal over time). First derivative functions allow calculation of the maximal velocity of fluorescent intensity (Vmax). Lag times (time from baseline intensity to onset of slope phase) and times to MA and Vmax are recorded. Area under the curve (AUC) for the first 2 hours of the reaction is also measured. This time point was chosen based upon the determination in developmental work that measurement of AUC at later time points (including end of assay) did not increase analytical sensitivity or precision.

In accordance with the objective of measuring relative changes in thrombin and plasmin generation capacity, rather than precise amounts of thrombin and plasmin generated, all assay parameters are reported as a percentage of corresponding values for the assay standard (adult pooled normal plasma, FACT, George King Bio-Medical Inc., Overland Park, Kans.) run with each plate. The normal control consisted of plasma from a pool of healthy individuals distinct from that of the assay standard. Plasma from a well characterized patient with severe factor VIII deficiency served as the abnormal coagulation control while a 20% plasminogen deficiency plasma was prepared as an abnormal fibrinolysis control from plasminogen deficient plasma with <1% activity mixed with pooled normal plasma.

### Normal Values for Thrombin and Plasmin Generation Parameters in Adult Plasma

Physiologic ranges for thrombin and plasmin parameters were determined in 20 healthy adults (Individual Adult CORE Set, George King Biomedical, Overland Park, Kans.). Table 1A provides median thrombin parameter values for T_{lag}, MA, Tₘₐₓ, V₁ₘₐₓ, Tv₁ₘₐₓ, AUC over 2 hours while Table 1B provides median plasmin parameter values for T_{lag}, MA, Tₘₐₓ, v₁ₘₐₓ, AUC over 2 hours for each of the 20 subjects.

Additional data was gathered in a 30 subject study, reflected in Table 1C.

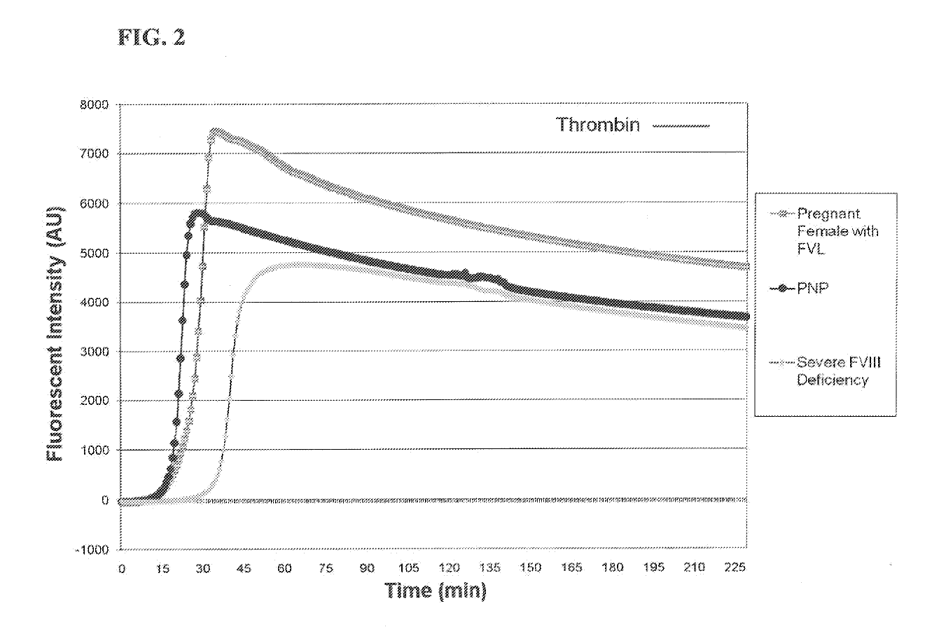

FIG. 2 represents an example of velocity of thrombin generation as curves from a pregnant woman heterozygous for factor V Leiden (hypercoagulable state) and a man with severe factor VIII deficiency (hypocoagulable state), both in comparison to pooled normal plasma of healthy non-pregnant adults

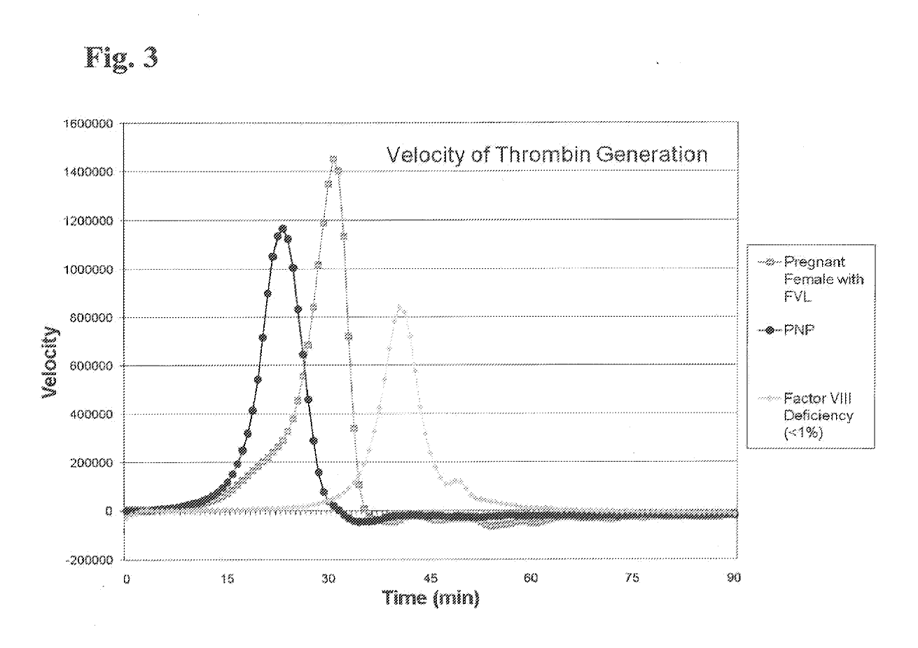

FIG. 3. represents velocity of thrombin generation as curves from a pregnant woman heterozygous for factor V Leiden (hypercoagulable state) and a man with severe factor VIII deficiency (hypocoagulable state), both in comparison to pooled normal plasma of healthy non-pregnant adults.

### Example 2

### Abnormal/Altered Plasma Experiments

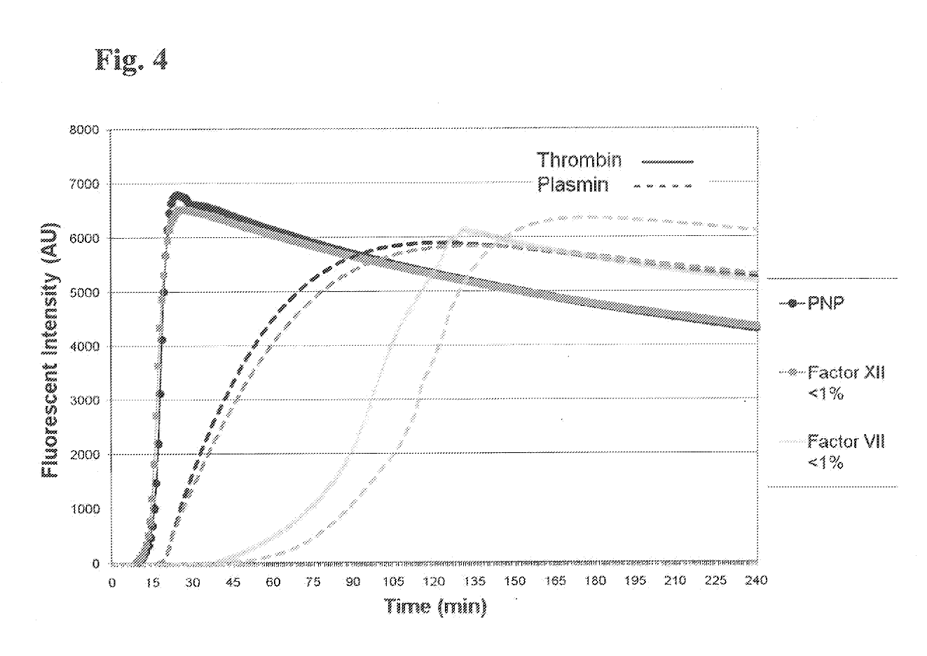

As represented in part in FIG. 4, Factor VIII and IX deficient plasmas were obtained from patients with severe congenital deficiency (factor VIII activity of <1 U/dL and factor IX activity <1 U/dL, respectively). Other specific factor-deficient human plasmas were obtained commercially as frozen specimens from patients with congenital factor deficiencies (factor I [fibrinogen], V, VII, X, XI, XII, XIII, prekallikrein, and high-molecular-weight kininogen [HMWK], George King Bio-Medical Inc., Overland Park, Kans.). Additionally, plasmas immunodepleted of factor II (Haematologic Technologies Inc, Essex Junction, Vt.), plasminogen, and alpha-2-antiplasmin (Affinity Biologicals) were tested. The activity level of the deficient factor was assayed at <1 U/dL in all cases, with the exception of fibrinogen concentration, which was <15 mg/dL. To inhibit fibrinolysis, standard normal pooled plasma was mixed with aminocaproic acid to achieve final plasma concentrations of 2.5 mg/mL and 0.25 mg/mL. In heparin studies, porcine unfractionated heparin sodium (APP Pharmaceuticals, LLC, Schaumburg, Ill.) was added to standard normal pooled plasma to achieve final plasma heparin concentrations of 0.5 U/mL, 0.1 U/mL, 0.05 U/mL, and 0.025 U/mL. For heparin reversal and the heparinase control, 5 mg of heparinase (Dade® Hepzyme®, Siemens, Plainfield, Ind.) was dissolved in 0.2 mL of plasma sample, as previously described.

To test the analytic sensitivity of the assay to prothrombin, fibrinogen, plasminogen, factor VIII and alpha-2-antiplasmin concentrations, deficient plasma of each was mixed with standard normal pooled plasma. Activities of 50%, 30%, 10%, and <1% were targeted for prothrombin, plasminogen, and alpha-2-antiplasmin mixing studies. Activities of 50%, 12.5%, 3.125%, and <1% of factor VIII and 350 mg/dL, 120 mg/dL, 30 mg/dL, and 15 mg/dL of fibrinogen were achieved in the respective mixing studies.

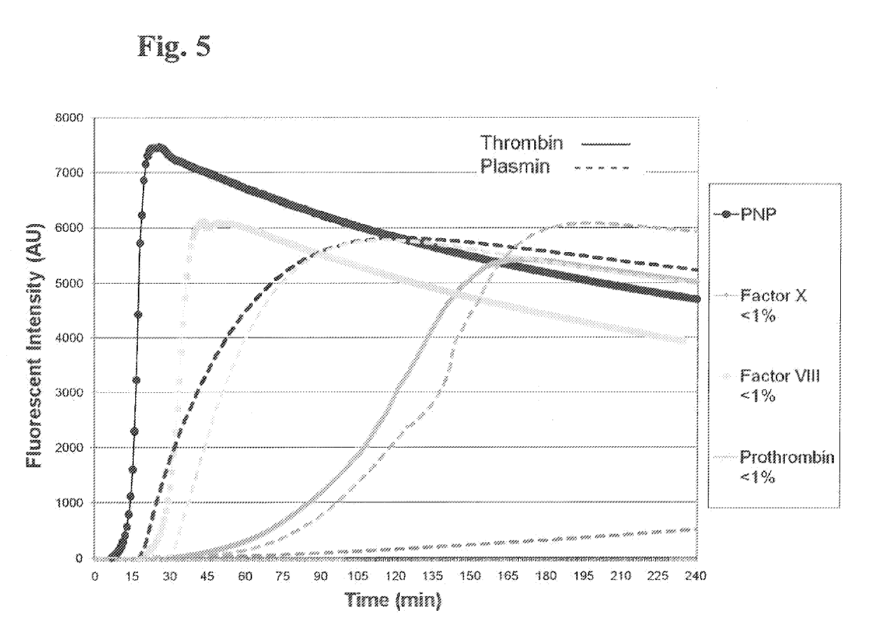

Influence of abnormalities in coagulation and fibrinolysis upon thrombin and plasmin generation capacity was investigated via STP assay application in representative examples of isolated factor deficiencies affecting the initiation of thrombin generation (FIG. 4), propagation of thrombin generation (FIG. 5), and fibrinolysis (FIG. 7). Data on lag times, MAs, and maximal velocities of thrombin and plasmin generation are represented in Table 3 for these altered coagulative and fibrinolytic states

### Correlative Laboratory Assay Procedures

PT was performed using TriniCLOT PT Excel, and aPTT using 0.025 molar calcium chloride and TriniCLOT Automated aPTT reagent (Trinity Biotech, Bray, Co. Wicklow, Ireland). Plasma fibrinogen concentration was determined by the clotting method of Clauss using Dade Behring thrombin and calibration reagents (Dade Behring, Marburg, Germany). These clotting assays were performed on the ST4 coagulometer (Diagnostica Stago, Asnieres-sur-Seine, France).

### Statistical Analysis

Distributions of data for each STP parameter in healthy children and adults were evaluated and reported as medians with ranges, based upon the non-parametric distribution of data in most cases. Upper and lower limits of normal values were established using mean +/−2*(standard deviation) when non-parametric data were normalized by log-transformation, and using median +/−1.5*(interquartile range) when data remained non-parametric following transformation. Distributions of values for STP parameters were compared between healthy children and adults by Mann-Whitney U test. P-values less than 0.05 were considered to be statistically significant. All analyses employed R statistical software, version 2.10.1

### Example 3

Tables 2A and 2B, illustrate examples of thrombin and plasmin generation parameters, for numerous experiments. Plasmas with severe deficiencies demonstrated decreased thrombin generation with low factor VIII, decreased plasmin with low factor I and aplasminogenemia, increased thrombin in a pregnant female with factor V Leiden, and increased plasmin in alpha-2-antiplasmin deficient state. These exemplary analytical techniques illustrate results of altered coagulation and fibrinolysis studies.

Any example may be modified according to procedures known to those skilled in the art, and are not meant to be either exhaustive or limiting. The following manuscripts and patent application are incorporated by reference in their entirety for all purposes: Goldenberg et al. Thromb Res 2005; 116(4):345-356; Goldenberg et al. Haemophilia 2006; 12(6):605-614; Goldenberg et al. Haemophilia 2008; 14(1):68-77; and Goldenberg et al. U.S. patent application Ser. No. 11/575,853, 371, National Stage 371 filing date Feb. 11, 2008.

TABLE 1A
Median (and observed range) thrombin generation parameters in healthy adults.

| | | | | | | AUC over |
|---|---|---|---|---|---|---|
| | T_{lag} | MA | Tₘₐₓ | V₁ₘₐₓ | Tv₁ₘₐₓ | 2 hrs |
| | (% Normal) | (% Normal) | (% Normal) | (% Normal) | (% Normal) | (% Normal) |
| Normal Adult | 99.5 | 106.5 | 94.8 | 109.5 | 98.2 | 106.3 |
| Mean (n = 20) | [73-148] | [96-116] | [69-138] | [75-154] | [78-135] | [93-115] |
| [observed range] | | | | | | |

TABLE 1B
Median (and observed range) plasmin generation parameters in healthy adults.

| | | | | | | AUC over |
|---|---|---|---|---|---|---|
| | T_{lag} | MA | Tₘₐₓ | V₁ₘₐₓ | Tv₁ₘₐₓ | 2 hrs |
| | (% Normal) | (% Normal) | (% Normal) | (% Normal) | (% Normal) | (% Normal) |
| Normal Adult | 99.6 | 105.8 | 104.9 | 107.2 | 100.5 | 104.2 |
| Mean (n = 20) | [86-123] | [96-116] | [91-164] | [74-145] | [85-127] | [84-118] |
| [observed range] | | | | | | |

TABLE 1C
Table 1: Intra- and Inter-assay Coefficients of Variation

| | (%) | | | | | |
|---|---|---|---|---|---|---|
| | Intra-assay CV | | | Inter-assay CV | | |
| | | | Plasminogen | | | Plasminogen |
| | Normal | F VIII <1% | Def 20% | Normal | F VIII <1% | Def 20% |
| | control | Control | Control | Control | Control | Control |
| | (n = 15) | (n = 13) | (n = 9) | (n = 17) | (n = 10) | (n = 10) |
| Lag_{T} | 3.2 | 3.4 | 4.8 | 10.2 | 10.5 | 12.1 |
| MA_{T} | 2.6 | 3.0 | 2.3 | 3.5 | 3.2 | 3.3 |
| V_{T}max | 12.4 | 11.2 | 6.2 | 18.5 | 14.3 | 14.4 |
| AUC_{T} 1^{st} | 3.6 | 3.8 | 5.0 | 2.0 | 2.9 | 1.2 |
| Deriv | | | | | | |
| Lag_{P} | 2.4 | 1.5 | 3.2 | 2.5 | 6.3 | 3.8 |
| MA_{P} | 4.1 | 4.2 | 5.3 | 3.5 | 6.1 | 5.4 |
| V_{P}max | 5.0 | 8.9 | 6.1 | 7.1 | 12.4 | 13.6 |
| AUC_{P} 1^{st} | 4.5 | 4.4 | 5.1 | 3.5 | 5.1 | 4.6 |
| Deriv | | | | | | |

Abbreviations: CV: coefficient of variation; FVIII: factor VIII; def: deficient; Lag_{T}: lag time to thrombin generation; MA_{T}: maximum amplitude of thrombin generation; V_{T}max: maximum velocity of thrombin generation; AUC_{T} 1^{st} Deriv: area under the first-derivative curve of thrombin generation; Lag_{P}: lag time to plasmin generation; MA_{P}: maximum amplitude of plasmin generation; V_{P}max: maximum velocity of plasmin generation; AUC_{P} 1^{st} Deriv: area under the first-derivative curve of plasmin generation.

Normal values for STP parameters were next determined in 30 healthy adults (median age 30 years; range: 19-54 years) and 91 children (median age 8 years; range: 1-17 years). Table 2 shows median values (with interquartile ranges [IQR]) for children versus adults. Distribution of STP values did not significantly differ among pediatric age groups evaluated, including ages 1-5 years (n=31), 6-11 years (n=31), and 12-17 years (n=29). Additionally, there were no significant differences between males and females. Hence, values were reported in aggregate for all pediatric ages and for both genders. Onset of both thrombin and plasmin generation was significantly delayed in children relative to adults. Additionally, maximal amplitudes of thrombin and plasmin generation were lower in children than adults. The velocity of thrombin generation was significantly lower in children than adults but the velocity of plasmin generation did not differ between these groups.

TABLE 2A
Table 2: Median Values (and Interquartile Ranges)
for Selected STP Parameters in Children and Adults

| | Pediatric | | | Adult | | | |
|---|---|---|---|---|---|---|---|
| Parameter | 25% | Median | 75% | 25% | Median | 75% | p-value |
| Thrombin Lag Time (% Nl) | 130.0 | 148.5 | 182.6 | 83.8 | 94.6 | 114.4 | <0.001 |
| Thrombin MA(% Nl) | 93.0 | 95.8 | 100.7 | 102.0 | 106.9 | 109.0 | <0.001 |
| Thrombin Vmax (% Nl) | 57.8 | 72.4 | 85.3 | 93.0 | 107.5 | 116.2 | <0.001 |
| Plasmin Lag Time (% Nl) | 126.0 | 135.5 | 148.4 | 88.5 | 97.1 | 105.0 | <0.001 |
| Plasmin MA(% Nl) | 94.4 | 100.4 | 105.5 | 100.8 | 104.9 | 108.8 | 0.005 |
| Plasmin Vmax (% Nl) | 85.5 | 95.9 | 107.9 | 90.7 | 106.0 | 113.1 | 0.14 |

Abbreviations: Vmax: maximum velocity; MA: maximum amplitude; Nl: normal (pooled normal adult plasma).

TABLE 2B
Thrombin generation parameters in each of the altered
states of coagulation/fibrinolysis shown in FIGS. 4-9.

| | | | | | | AUC over |
|---|---|---|---|---|---|---|
| | T_{lag} | MA | Tₘₐₓ | V₁ₘₐₓ | Tv₁ₘₐₓ | 2 hrs |
| | (% Normal) | (% Normal) | (% Normal) | (% Normal) | (% Normal) | (% Normal) |
| Normal Adult | 99.5 | 106.5 | 94.8 | 109.5 | 98.2 | 106.3 |
| Mean (n = 20) | [73-148] | [96-116] | [69-138] | [75-154] | [78-135] | [93-115] |
| [observed range] | | | | | | |
| Pregnancy with | 110.3 | 117 | 110.8 | 123 | 131.7 | 109 |
| factor V Leiden | | | | | | |
| A2AP (0%) | 72.5 | 107 | 124.1 | 46 | 112.9 | 106 |
| Factor VIII (50%) | 104.8 | 100 | 102.6 | 100 | 113.1 | 98 |
| Factor VIII (12.5%) | 142.9 | 98 | 137.2 | 84 | 141 | 98 |
| Factor VIII (3%) | 171.4 | 90 | 175.6 | 66 | 154.1 | 92 |
| Factor VIII (<1%) | 204.8 | 81 | 193.6 | 59 | 173.8 | 86 |
| Fibrinogen (120 mg/dL) | 72.7 | 99 | 74.7 | 116 | 80.3 | 101 |
| Fibrinogen (26 mg/dL) | 68.2 | 98 | 70.1 | 113 | 75.8 | 99 |
| Fibrinogen (15 mg/dL) | 65.9 | 96 | 67.8 | 115 | 72.7 | 97 |
| Plasminogen (50%) | 135 | 101 | 124.1 | 91 | 126.1 | 99 |
| Plasminogen (30%) | 150 | 105 | 138.6 | 92 | 142 | 105 |
| Plasminogen (10%) | 195 | 95 | 177.1 | 67 | 172.5 | 97 |
| Plasminogen (0%) | 245 | 85 | 259 | 28 | 252.2 | 81 |

Results > 2 standard deviations beyond mean values in healthy adults are shown in red, underlined

TABLE 2C
Plasmin generation parameters in each of the altered states of coagulation/fibrinolysis shown in FIGS. 4-9.

| | | | | | | AUC over |
|---|---|---|---|---|---|---|
| | T_{lag} | MA | Tₘₐₓ | V₁ₘₐₓ | Tv₁ₘₐₓ | 2 hrs |
| | (% Normal) | (% Normal) | (% Normal) | (% Normal) | (% Normal) | (% Normal) |
| Normal Adult | 99.6 | 105.8 | 104.9 | 107.2 | 100.5 | 104.2 |
| Mean (n = 20) | [86-123] | [96-116] | [91-164] | [74-145] | [85-127] | [84-118] |
| [observed range] | | | | | | |
| Pregnancy with | 129.4 | 108 | 111.6 | 88 | 120.7 | 101 |
| factor V Leiden | | | | | | |
| A2AP (0%) | 60.9 | 116 | 59.8 | 304 | 89.3 | 129 |
| Factor VIII (50%) | 111.8 | 98 | 100 | 94 | 112.8 | 97 |
| Factor VIII (12.5%) | 138.2 | 102 | 99.7 | 92 | 143.6 | 102 |
| Factor VIII (3%) | 151.5 | 96 | 100.6 | 83 | 156.4 | 95 |
| Factor VIII (<1%) | 163.2 | 92 | 100.3 | 80 | 175.6 | 91 |
| Fibrinogen (120 mg/dL) | 87.8 | 100 | 106.6 | 73 | 138.5 | 94 |
| Fibrinogen (26 mg/dL) | 118.9 | 82 | 177.3 | 29 | 167.9 | 54 |
| Fibrinogen (15 mg/dL) | 148.6 | 62 | 177.3 | 18 | 175.6 | 38 |
| Plasminogen (50%) | 126.4 | 91 | 179.3 | 72 | 125.6 | 66 |
| Plasminogen (30%) | 168.1 | 57 | 179.3 | 31 | 162.8 | 35 |
| Plasminogen (10%) | 330.6 | 21 | 179.3 | 6 | 487.2 | 15 |
| Plasminogen (0%) | 0 | 0 | 178.4 | 2 | 1.3 | 0 |

Results > 2 standard deviations beyond mean values in healthy adults are shown in red, underlined.

TABLE 3
Table 3: Representative STP Values for Severe Deficiency States, Relative to the Assay Standard (Pooled Normal Adult Plasma)

| | | | | Thrombin | | | | Plasmin |
|---|---|---|---|---|---|---|---|---|
| | Thrombin | Thrombin | Thrombin | AUC x | Plasmin | Plasmin | Plasmin | AUC x |
| | Lag Time | MA | Vmax | 2 hrs | Lag Time | MA | Vmax | 2 hrs |
| | (% Normal) | (% Normal) | (% Normal) | (% Normal) | (% Normal) | (% Normal) | (% Normal) | (% Normal) |
| Normal Adult | 94.6 | 106.9 | 107.5 | 106.4 | 97.1 | 104.9 | 106 | 103.7 |
| Median (n = 30) | [70-144] | [86-116] | [66-139] | [61-118] | [60-121] | [90-116] | [73-134] | [79-121] |
| [LLN, ULN] | | | | | | | | |
| Severe | | | | | | | | |
| Deficiencies: | | | | | | | | |
| Fibrinogen | 86 | 99 | 119 | 100 | 650* | 11* | 3* | 1* |
| Prothrombin | 0* | 1* | 0* | 0* | 553* | 8* | 2* | 1* |
| Factor V | 0* | 2* | 0* | 0* | [symbol] * | 21* | 6* | 4* |
| Factor X | 465* | 81* | 7* | 10* | 327* | 108 | 137* | 12* |
| Factor VIII | 306* | 81* | 59* | 72* | 183* | 92 | 80 | 70* |
| Factor IX | 315* | 74* | 28* | 54* | 225* | 94 | 63* | 48* |
| Factor XI | 121 | 95* | 73 | 96 | 116 | 96 | 61* | 78* |
| Factor XII | 84 | 95* | 83 | 99 | 96 | 98 | 81 | 93 |
| Factor VII | 435* | 91* | 11* | 34* | 367* | 104 | 125 | [symbol] * |
| Factor XIII | 100 | 92* | 100 | 92 | 117 | 84* | 55* | 65* |
| Plasminogen | 228* | 97 | 54* | 80* | 0* | 0* | 1* | 0* |
| α2AP | 73 | 107 | 46* | 108 | 61* | 116 | 304* | 150* |

[symbol]

indicates data missing or illegible when filed

Abbreviations: Vmax: maximum velocity; MA: maximum amplitude, AUC×2 hrs=area under the curve over 2 hours, LLN: lower limit of normal; ULN: upper limit of normal; α2AP: alpha-2-antiplasmin. For methods by which LLN and ULN were calculated, see Methods.

### Example 4

The influence of abnormalities in coagulation and fibrinolysis upon thrombin and plasmin generation capacity was investigated via STP assay application in representative examples of isolated factor deficiencies affecting the initiation of thrombin generation (FIG. 4), propagation of thrombin generation (FIG. 5), and fibrinolysis (FIG. 7). Data on lag times, MAs, and maximal velocities of thrombin and plasmin generation are summarized in Table 3 for these altered coagulative and fibrinolytic states.

FIG. 4 illustrates STP curves for severe deficiencies (<1% activity) of selected factors involved in initiation of thrombin generation, including factors XII and VII, relative to pooled normal adult plasma (PNP).

FIG. 5 illustrates STP curves for severe deficiencies (<1% activity) of selected factors involved in propagation of thrombin generation, including factor X, factor VIII, and prothrombin, relative to pooled normal adult plasma (PNP).

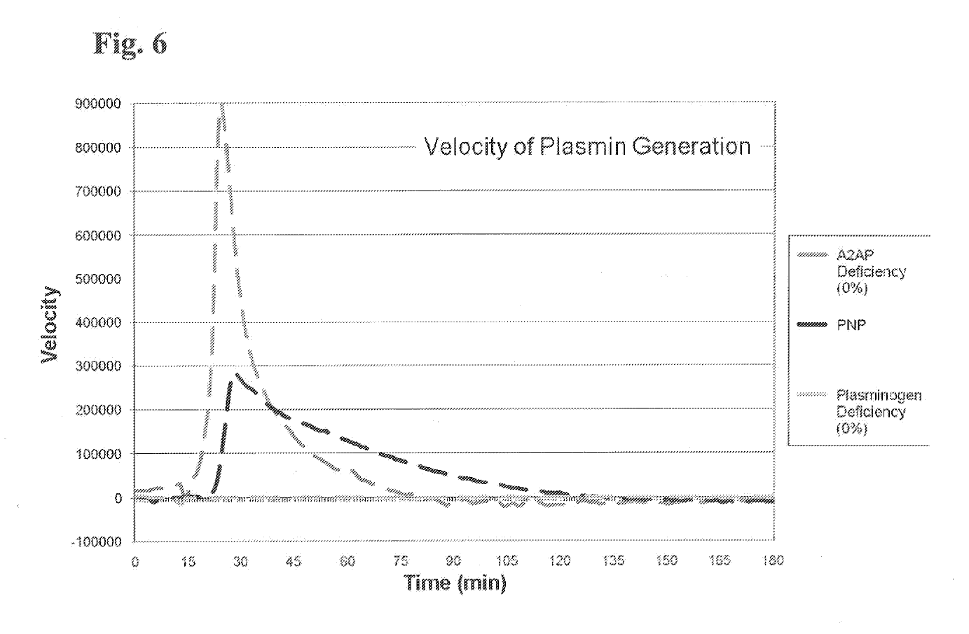

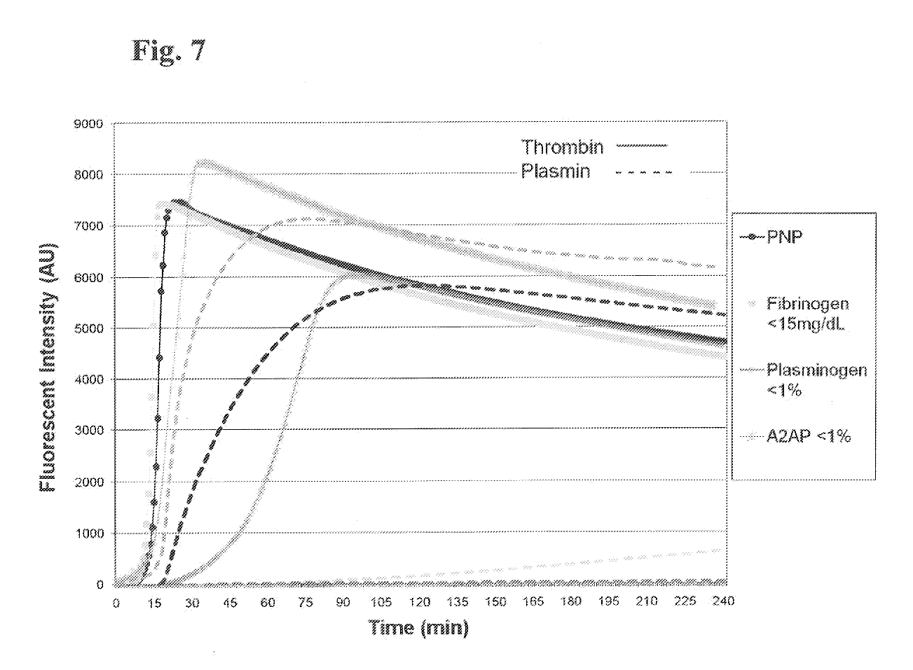

FIG. 6. represents velocity of plasmin generation curves of plasma from alpha-2-antiplasmin deficient state (hyperfibrinolytic state) and plasminogen deficient state (hypofibrinolytic state), both in comparison to pooled normal plasma of healthy adults.

FIG. 7 illustrates STP curves for defects of fibrinolysis, including afibrinogenemia (fibrinogen <15 mg/dL [Clauss method]), severe plasminogen deficiency (<1%), and severe alpha-2-antiplasmin (A2AP) deficiency (<1%), relative to pooled normal adult plasma (PNP).

### Example 5

### Effect of Deficiencies of Coagulation Factors and Fibrinolytic Regulators on Thrombin and Plasmin Generation Curves and Parameters

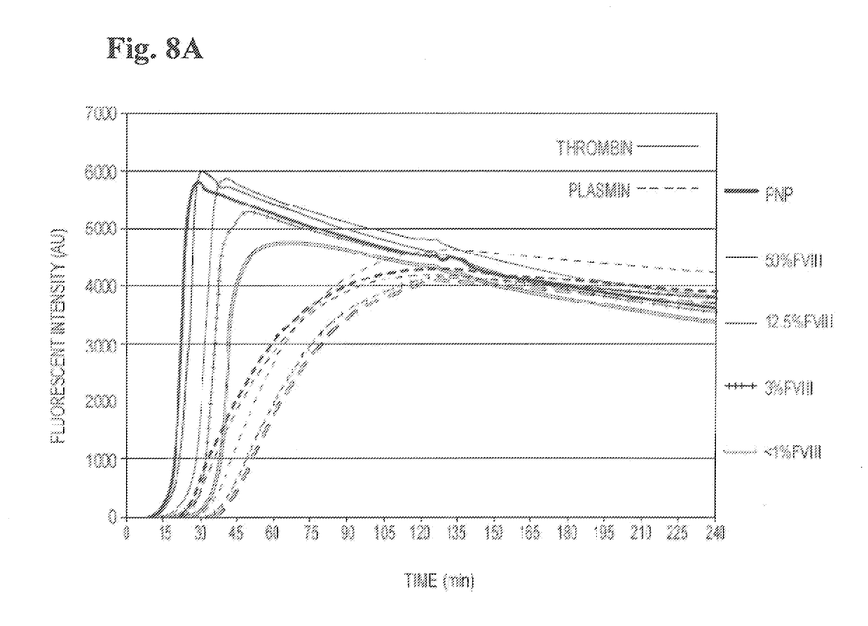

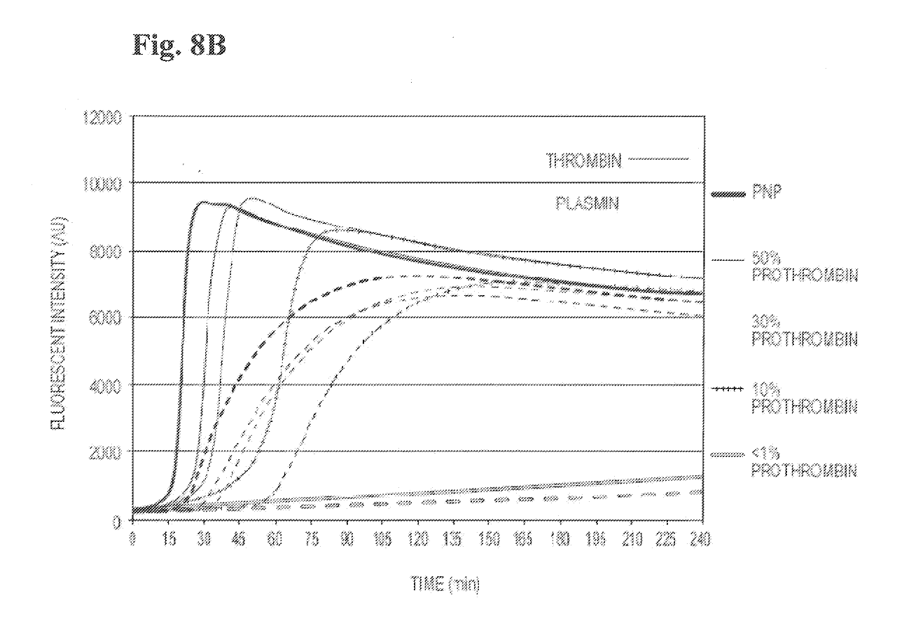

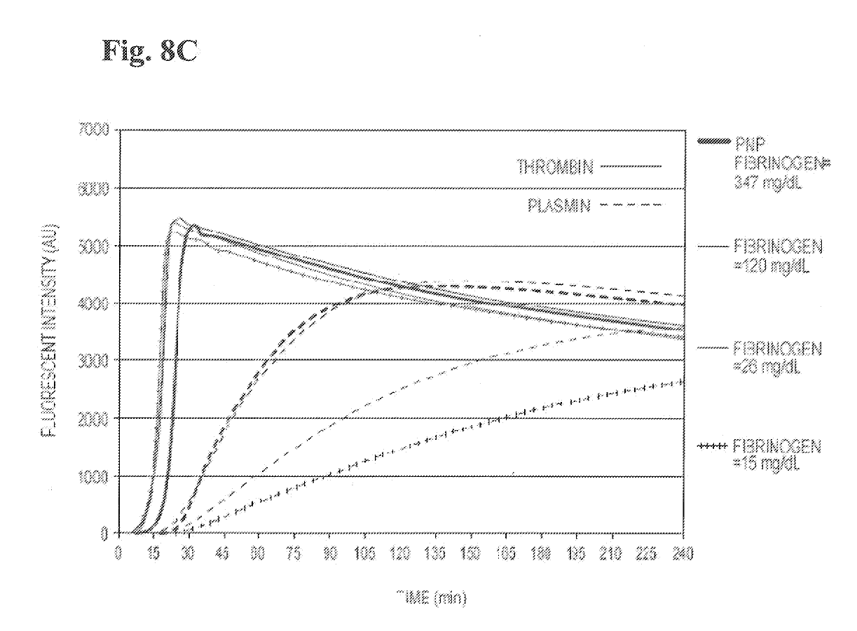

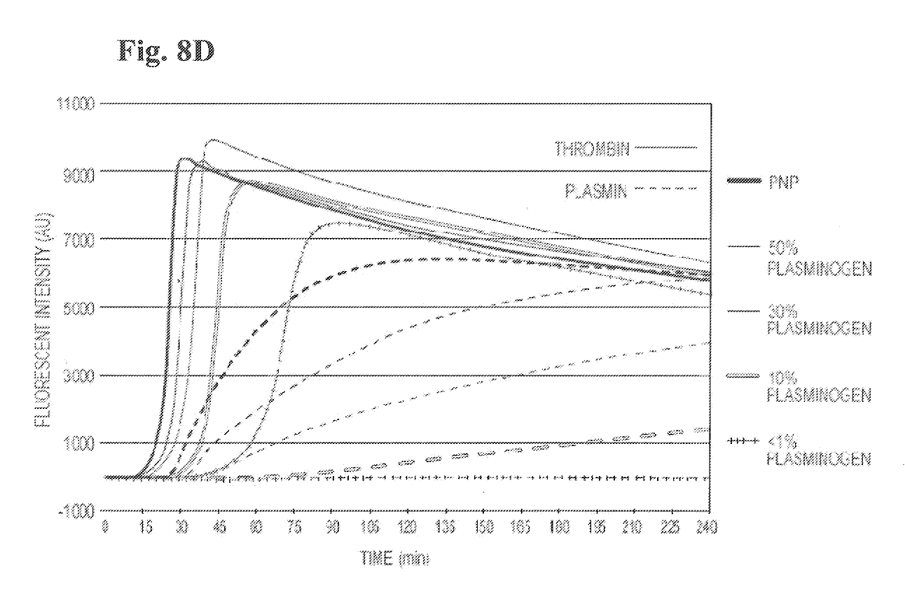

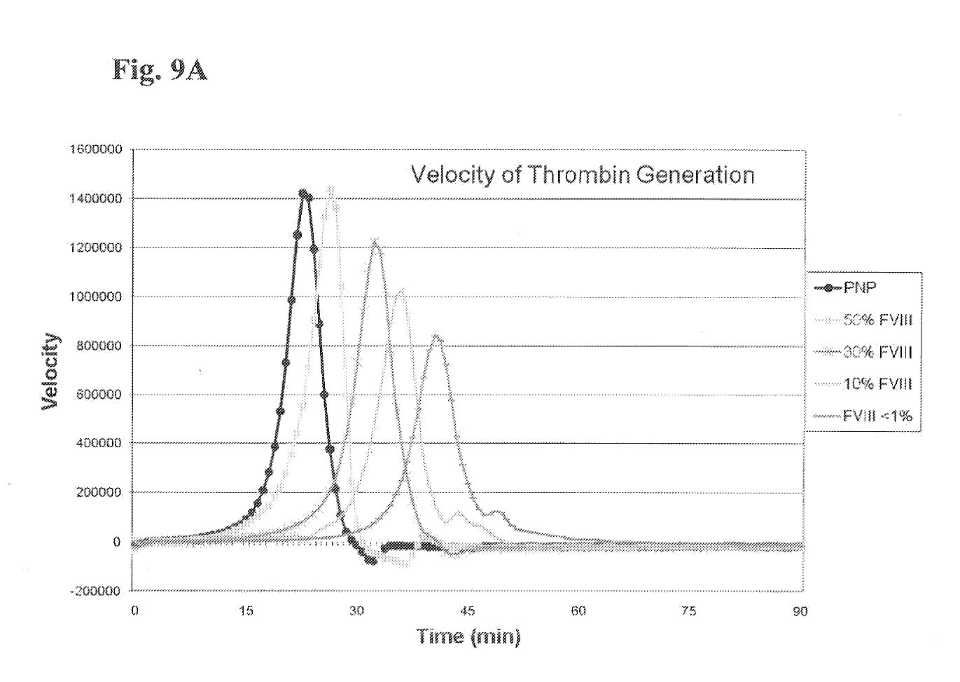

FIGS. 8A-8D illustrate mixing studies demonstrating concentration-dependence of STP to selected factor deficiencies, including: (A). Factor VIII; (B). Prothrombin; (C). Fibrinogen; and (D). Plasminogen.

It was observed that thrombin generation capacity was reduced in factor VII deficiency but was normal in factor XII deficiency. Contact pathway inhibition via the addition of corn trypsin inhibitor to citrate whole blood collection tubes did not alter STP findings in normal plasma in these experiments, relative to absence of corn trypsin inhibitor (data not shown). While tenase component (factors VIII and IX) deficiencies reduced thrombin generation, the most profound impairment was observed for common pathway factor deficiencies, other than afibrinogenemia (FIG. 7), where it was observed that thrombin generation was not reduced. In vitro experiments demonstrated concentration-responsiveness of thrombin generation capacity to deficiency of factor VIII, fibrinogen, and prothrombin (mixing studies illustrated in FIG. 8) and to exogenous TF (data not shown).

Plasmin generation capacity in afibrinogenemia was markedly impaired, consistent with the importance of fibrin as a scaffold for normal plasmin generation. Plasmin generation was also severely impaired in plasminogen deficiency (FIG. 8D) and with aminocaproic acid treatment of plasma (data not shown). Plasmin generation showed concentration-dependence to deficiency of plasminogen and fibrinogen (mixing studies in FIG. 5) and to exogenous tPA (data not shown). Plasmin generation was greatly enhanced by alpha-2-antiplasmin deficiency (FIG. 7) and excess tPA (data not shown).

Analytical sensitivity of thrombin and plasmin generation capacity to various concentrations of heparin was tested with both unfractionated heparin and the low molecular weight heparin dalteparin. Thrombin and plasmin generation by STP were sensitive to heparin concentrations as low as 0.05 U/mL, and the ablation of thrombin and plasmin generation observed at a concentration of 1 U/mL was completely reversed via heparinase pre-treatment of plasma (data not shown).

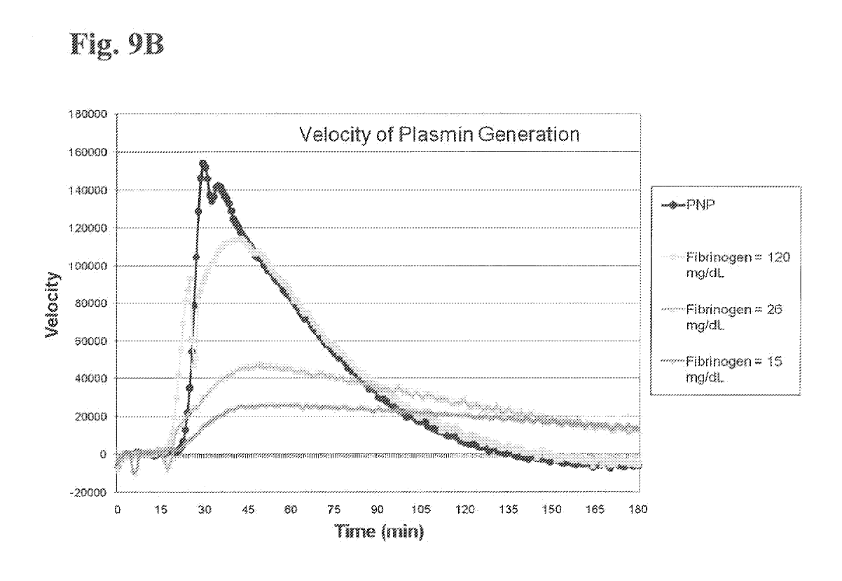

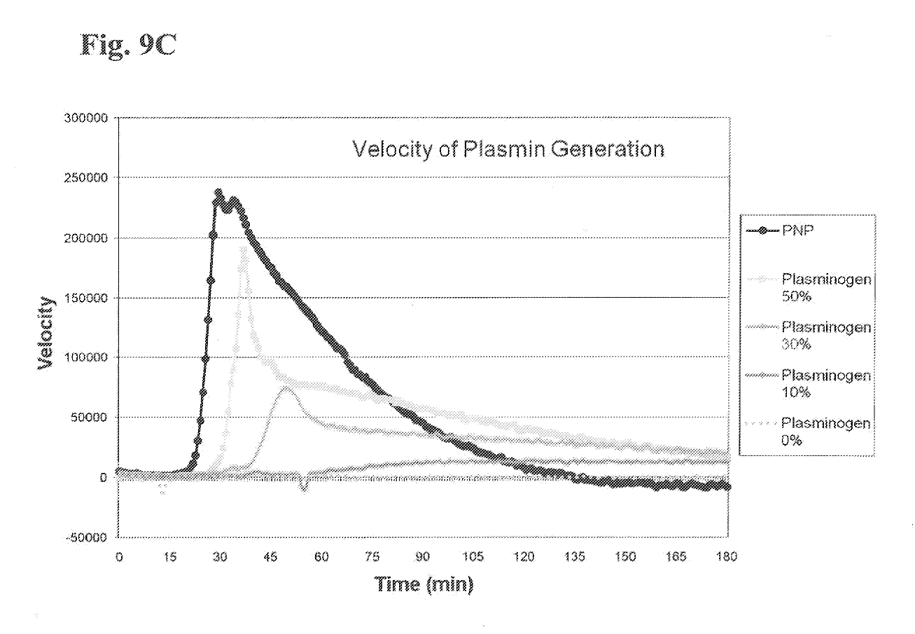

FIGS. 9A, 9B, and 9C. The influence of (A) factor VIII activity on the velocity of thrombin generation as a curve, (B) fibrinogen concentration on the velocity of plasmin generation as a curve, and (C) plasminogen concentration on the velocity of plasmin generation as a curve.

Some observations from STP curves in FIGS. 4, 5 and 7 illustrate potential for assays disclosed herein to provide novel understandings of coagulative and fibrinolytic mechanisms and raise several observations. (1) thrombin is generated first and is a prerequisite for plasmin generation. (2) Plasmin generation, but not thrombin generation, is dependent on concentrations of fibrinogen and plasminogen. (3) Prothrombinase complex components are critical for thrombin generation. (4) Tenase complex components factor VIII and factor IX, but not factor XII or other contact factors, appear necessary for enhancement of thrombin generation. (5) In the absence of plasmin generation (except in severe fibrinogen deficiency), thrombin generation is impaired. (6) In the absence of fibrinogen-fibrin (antithrombin I), normal thrombin generation (or possibly increased if considering it relative to the lack of plasmin generation) is observed.

Simultaneous measurement of thrombin and plasmin generation capacity in parallel wells of assays disclosed herein renders these techniques clinically useful for assessment of both coagulative and fibrinolytic potential, and can provide an immediate interpretation of overall hemostatic balance. The lack of requirement for contact pathway inhibition during specimen collection (attributable to the 5 pM concentration of TF employed in the assay, as previously described) simplifies procedural logistics in the clinical and clinical research settings, and avoids the confounding influence of corn trypsin inhibitor upon thrombin generation measurements in the presence of tPA. The reporting of results as a percent of measured values for the assay standard (pooled normal adult plasma) not only reduces the impact of inter-assay variation in measurements, but also minimizes the dependence of assay findings upon inner filter effects, intrinsic levels of alpha-2-macroglobulin, specific reagent concentrations and assay conditions, which is engendered by methods seeking to quantify molar amounts of thrombin or plasmin generated. For at least these reasons, STP assays are foreseen to have vast utility as a clinical and clinical research and diagnostic tools.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1-5. (canceled)

6. An assay method comprising:
obtaining a sample from a subject;
adding a an exogenous buffered reactant solution to the sample, wherein the solution contains at least one activator of coagulation and at least one activator of clot lysis; and
simultaneously measuring both thrombin and plasmin generation in the sample.

7. The method of claim 6, wherein thrombin and plasmin capacities are measured.

8. The method of claim 6, wherein thrombin and plasmin generation are measured fluorometrically.

9. The method of claim 6, wherein thrombin and plasmin generation are measured continuously for a period of up to four hours.

10. The method of claim 6, wherein thrombin and plasmin generation are measured at frequent selected time intervals for a period of up to four hours.

11. The method of claim 10, wherein the time interval is selected from the group consisting of 5, 10, 15, 20, 25, 30, 35, 40, 45, 50, 55 and 60 seconds.

12. The method of claim 6, wherein the activator of coagulation is selected from the group consisting of calcium, tissue factor (TF), phospholipid reagent, platelet reagent, or a combination thereof.

13. The method of claim 6, wherein the activator of coagulation is TF and the concentration of TF is 1 pM to 10 pM.

14. The method of claim 13, wherein the concentration of TF is 5 pM.

15. The method of claim 6, wherein the activator of fibrinolysis is selected from the group consisting of tissue-type plasminogen activator (tPA), urokinase-type plasminogen activator (uPA, or urokinase), plasmin, potato tuber carboxypeptidase inhibitor, other carboxypeptidases or a combination thereof.

16. The method of claim 6, further comprising inhibiting contact activation of coagulation using corn trypsin inhibitor or other contact activation inhibitor.

17. The method of claim 6, further comprising adding heparin or heparin-like substances to the sample.

18. The method of claim 6, further comprising adding an activator of protein C pathway.

19-21. (canceled)

22. The method of claim 6, wherein the subject has or is suspected of having a heart condition.

23. The method of claim 6, wherein the subject has or is suspected of having an abnormal blood condition.

24-25. (canceled)

26. The method of claim 6, further comprising comparing thrombin and plasmin generation in a sample from a control subject with the sample from the subject wherein the subject has an abnormal blood condition or a heart condition.

27. A kit for analyzing thrombin and plasmin generation in a blood sample comprising:
a buffered reactant solution;
at least one activator of coagulation;
at least one activator of fibrinolysis; and
a device for measuring thrombin generation and plasmin generation.

28-35. (canceled)

36. The method of claim 12,wherein the tissue factor is lipidated tissue factor.

37. The method of claim 6, wherein the sample is pre-treated with heparinase.

38. The method of claim 7, wherein both plasmin and thrombin capacities are determined by rate, amplitude and amount of clot formed and lysed.

39-40. (canceled)

41. The method of claim 6, wherein the sample comprises a platelet-poor plasma sample.
